(19) United States
(12) Patent Application Publication
(10) Pub. No.: US 2007/0020313 A1
(43) Pub. Date: Jan. 25, 2007

(54) Composition of amylase inhibitors and fibers to reduce the absorption of starch in human digestion from starchy foods, and to reduce the glycemic index of starchy foods

(75) Inventors: Harvey William Palat, Freehold, NJ (US); Pete Maletto, Long Branch, NJ (US); Daniel A. Maiullo, Tinton Falls, NJ (US)

Correspondence Address: DANIEL MAIULLO, 2970 Shafto Rd, Tinton Falls, NJ, 07753, US

(73) Assignee: SUGAR SENSE INC, New Jersey Corporation, FREEHOLD, NJ (US)

(21) Appl. No.: 11/187,986

(22) Filed: Jul. 25, 2005

(51) Int. Cl. A61K 47/00; A61K 36/82; A61K 36/899

(52) U.S. Cl. 424/439; 424/729; 424/750

(57) ABSTRACT

A composition of amylase inhibitors and fibers to reduce the absorption of starch in human digestion from starch-containing foods, and to reduce the glycemic index of starch containing foods.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

There has been an increasing public awareness about the negative effects of consuming excessive starch, as well as the skyrocketing percentage of individuals who are diabetic in the United States and other countries as well. It has been estimated by the United States government that there are 18 million Americans that are currently diabetic. Many have reduced consumption of starchy foods to avoid excess elevations in blood sugar. Most people are looking to reduce starches because they are following some type of diet or weight loss regimen.

The number of people following low carbohydrate, low glycemic or starch and sugar reduced diets is on the rise, and may include as much as one-third of the population or higher. This number varies seasonally and typically is higher, for example, after the holidays at the end of the year. Although many people reduce starches to try to lose weight, the products on the marketplace designed with reduced starch ingredients have not lived up to the esthetic characteristics of the corresponding traditional food products.

The numbers of diabetics, pre diabetics and obese in America are on the rise, which is evident by the fact that America is generally regarded as the fattest country in the world due to Americans' indulgence in starches found in most foods. A common practice among individuals trying to reduce their intake of starches is to purchase products that have ingredients with reduced starch content. As would be expected, the primary emphasis in the development of an all natural, starch-reduced product is esthetics, since it is generally recognized that low starch products have little chance of survival in the marketplace because of most of these products have an intolerable taste. It will be appreciated that there is significant competition to achieve such products whose esthetic characteristics most closely resemble the corresponding traditional starchy foods.

Starchy ingredients such as wheat flours have the universal functionality of producing excellent taste and texture that Amercans are used to in their foods. Mainstream products using flour based formulations unfortunately result in high increases in blood sugar because of high starch content. It is generally agreed that there is need to reduce starch absorption without sacrificing taste as public awareness increases everyday about the effects of starchy foods. Of course the use of starch blockers has recently gained in popularity with the success and growth of carbohydrate restricted diets. One of these is an extract of the white kidney bean (Phaseolus vulgaris). There are many enzymes in the human digestive system which help digest the variety of foods we consume. The alpha amylase enzyme is responsible for breaking down starchy foods, such as breads, potatoes, rice, and pasta into sugars that can be absorbed through the intestinal wall. Alpha amylase inhibitors are molecules which interfere with the action of the alpha amylase enzyme. The present invention provides a novel, all natural, high fiber, amylase inhibitor composition that reduces starch at the point of digestion, and speeds the starch transit time of the food in the digestive tract. The development of a starch inhibiting compound along with needed fibers help speed transit time through the digestive tract, reducing starch absorption even more, and producing a low glycemic impact on the consumer from the food product. This approach retains the ingredients in the product that produce the best esthetics.

### BRIEF SUMMARY OF THE INVENTION

This invention relates to a novel, all natural, high fiber, amylase inhibitor, low glycemic composition, consisting of maltodextrin fiber, oat fiber, wheat-based amylase inhibitor, and/or epigallocatechin gallate from green tea extract. Together in this formulation these natural agents reduce amylase starch absorption in human saliva and digestive processes, resulting in a lower impact on blood sugar, commonly known as a glycemic response, which is extremely beneficial to diabetics and those looking to reduce or stabilize blood sugar, whether for weight loss or other health benefits.

### DETAILED DESCRIPTION OF THE INVENTION

In accordance with the present invention, there is provided an all natural composition for the reduction of starch digestion in humans. The ingredients of the subject composition are as follows.

The principal ingredient of the present invention is a soluble fiber of maltodextrin. This ingredient is a spray-dried powder produced by the pyrolysis and controlled enzymatic hydrolysis of cornstarch, is readily dispersible in water, and carries no inherent flavor. This soluble fiber ingredient, which is unique to this formulation, contributes solids and texture to any finished product. A preferred maltodextrin soluble powder product is Fibersol-2, available from Matsutani America, which contains from about 80% to 95% fiber. This material is present in the formulation from about 40% to about 60% by weight, preferably about 47.5% by weight.

The second fiber component is Oat Fiber. Oat Fiber is fiber extracted from oat bran and purified to a 95% fiber content. The functionality in the subject formulation is to increase transit time through the digestive tract. Along with the amylase inhibitors this combination reduces starch absorption into the bloodstream, thereby decreasing the glycemic index of the food into which the present invention has been incorporated. This material is present in the formulation from about 20% to about 30% by weight, preferably about 25% by weight. Oat Fiber 300-33 is available as Sun Opta from Canadian Harvest.

The present invention contains an amylase inhibitor from wheat, which functions to decrease amylase (starch digesting enzyme) effectiveness in human saliva and digestive processes. This ingredient reduces starch absorption. This quality allows the use of starch-containing ingredients in food that provide great taste and texture, but also reduces their digestion in the human body thereby reducing any increase in blood sugar resulting from consumption of the food. This amylase inhibitor also reduces the glycemic index of the food due to its inhibiting effects on starch digestion. This fact should prove attractive to those with special diet considerations or who are diabetic. A preferred wheat-based amylase inhibitor is Alpha Trim, available from Nutricepts Inc. It is used in the present invention in from about 20 percent to about 30 percent by weight, preferably about 25 percent by weight.

The present invention contains another amylase inhibitor from green tea extract called epigallocatechin gallate (EGCG). This pure EGCG compound has been clinically shown to block digestion of carbohydrates by interfereing with amylase effectiveness. Preferably, the subject formulation contains EGCG as Sunphenon from Taiyo International having a catechin content of about 90 percent. Sunphenon EGCG is present in the subject formulations from about 1 percent to about 10 percent by weight, preferably about 2.5 percent by weight.

It has been found that the ingredients enumerated above complement each other in developing a starch inhibiting formulation that occurs through anti amylase activity and decreased starch transit time due to added fiber. It is also not conventional to add this combination of amylase inhibitors with high fiber components to any food product. The present invention may be utilized in all varieties of solid and semi-solid food preparations, such as potato preparations and breads, where starch would normally be utilized.

The formulation of the present invention can be incorporated into any food where inhibiting starch absorption is desired in accordance with traditional recipes. It allows for similar functionality to starch in regards to taste and texture in baked goods, while providing solids content in the overall product.

The following example further illustrates the current invention, but is not in any way intended as being limiting thereon:

### EXAMPLE 1

The following formulation was utilized to prepare the starch inhibiting composition bulk powder:

Ingredient Quantity in Grams: Maltodextrin Fiber 475.0 (Fibersol 2, Matsutani America), Oat Fiber (Sun Opta 300-33) 250.0, Alpha Trim 250.0, EGCG 25.0, Total 1,000.

The ingredients consisting of the oat and maltodextrin dietary fiber, amylase inhibitor from wheat, and amylase inhibitor from green tea, are then combined in a suitable mixing vessel and mixed until thoroughly blended.

The present invention provides a number of advantages, such as the following:

1. Oat and maltodextrin fiber act as soluble fiber bulking agents in the invention that speed starch digestion in the digestive tract. Like all dietary fibers, they are not digested in the stomach or small intestine but decrease transit time. However, because they are completely fermented in the colon, they contribute to better gut function. They speed starch transit through the gut, reducing its digestion, improving regularity, and reducing constipation.

2. The all natural ingredients of the invention have no history of side effects, are non-carcinogenic and non-toxic.

3. The present invention permits the usage of high starch ingredients that provide great taste and texture while inhibiting their digestion.

4. Like all dietary fibers, oat and maltodextrin fiber have no impact on blood sugar. These fibers in the subject formulation also have the ability to reduce the glycemic index of any food eaten, which makes it suitable for special diets where glycemic control is necessary, such as diabetes.

5. Unlike other amylase inhibitors, the present invention of all natural ingredients with fiber inhibits starch digestion in human saliva. Others, such as extract of the white kidney bean (Phaseolus vulgaris), do not inhibit amylase in human saliva.

What is claimed is:

1. A composition that provides a reduction in starch digestion in humans comprising any two or more of the following: a) all natural wheat-based amylase inhibitor, b) all natural green tea extract with EGCG content; c) all natural oat fiber; d) all natural digestion resistant fiber from maltodextrin.

2. A composition according to claim 1, for use in solid and/or semi-solid foods.

3. A composition according to claim 1, for use as an ingredient to decrease the glycemic index of solid and/or semi-solid foods.

4. A composition according to claim 1, comprising: a) all natural amylase inhibitors derived from wheat; b) all natural epigallocatechin gallate derived from green tea extract; c) all natural fibers derived from oat fiber; d) all natural digestion resistant fiber derived from maltodextrin fiber.

5. A composition according to claim 4, for use in solid and/or semi-solid foods.

6. A composition according to claim 4, for use as an ingredient to decrease the glycemic index of solid and/or semi-solid foods.

7. A composition according to claim 1, comprising: a) all natural amylase inhibitors derived from wheat in the amount of approximately 20 percent to 30 percent of the total composition by weight; b) all natural epigallocatechin gallate derived from green tea extract in the amount of approximately 1 percent to 10 percent of the total composition by weight; c) all natural fibers derived from oat fiber in the amount of approximately 20 percent to 30 percent of the total composition by weight; d) all natural digestion resistant fiber derived from maltodextrin fiber in the amount of approximately 40 percent to 60 percent of the total composition by weight.

8. A composition according to claim 7, for use in solid and/or semi-solid foods.

9. A composition according to claim 7, for use as an ingredient to decrease the glycemic index of solid and/or semi-solid foods.
